SEQUENCE LISTING (36 sequences)

SEQ ID NO: 1 - DNA, 72 bp, synthetic construct
  source 1..72: mol_type = other DNA
ggaggctctcgggacgacnnnnnnnnnnnnnnnnnnnnnnnnnnnnnngtcgtcccgatgctgcaatcgtaa

SEQ ID NO: 2 - DNA, 18 bp, synthetic construct
  source 1..18: mol_type = other DNA
ggaggctctcgggacgac

SEQ ID NO: 3 - DNA, 18 bp, unidentified
  source 1..18: mol_type = other DNA
gtcgtcccgagagccata

SEQ ID NO: 4 - DNA, 22 bp, synthetic construct
  source 1..22: mol_type = other DNA
ttacgattgcagcatcgggacg

SEQ ID NO: 5 - DNA, 55 bp, synthetic construct
  source 1..55: mol_type = other DNA
tcgtcggcagcgtcagatgtgtataagagacagccatggaggctctcgggacgac

SEQ ID NO: 6 - DNA, 55 bp, synthetic construct
  source 1..55: mol_type = other DNA
tcgtcggcagcgtcagatgtgtataagagacagccaaggaggctctcgggacgac

SEQ ID NO: 7 - DNA, 55 bp, synthetic construct
  source 1..55: mol_type = other DNA
tcgtcggcagcgtcagatgtgtataagagacagcgatggaggctctcgggacgac

SEQ ID NO: 8 - DNA, 55 bp, synthetic construct
  source 1..55: mol_type = other DNA
tcgtcggcagcgtcagatgtgtataagagacagcgttggaggctctcgggacgac

SEQ ID NO: 9 - DNA, 55 bp, synthetic construct
  source 1..55: mol_type = other DNA
tcgtcggcagcgtcagatgtgtataagagacagatacggaggctctcgggacgac

SEQ ID NO: 10 - DNA, 55 bp, synthetic construct
  source 1..55: mol_type = other DNA
tcgtcggcagcgtcagatgtgtataagagacagatccggaggctctcgggacgac

SEQ ID NO: 11 - DNA, 62 bp, synthetic construct
  source 1..62: mol_type = other DNA
gtctcgtgggctcggagatgtgtataagagacagcctattacgattgcagcatcgggacgac

SEQ ID NO: 12 - DNA, 62 bp, synthetic construct
  source 1..62: mol_type = other DNA
gtctcgtgggctcggagatgtgtataagagacagccttttacgattgcagcatcgggacgac

SEQ ID NO: 13 - DNA, 62 bp, synthetic construct
  source 1..62: mol_type = other DNA
gtctcgtgggctcggagatgtgtataagagacagcgtattacgattgcagcatcgggacgac

SEQ ID NO: 14 - DNA, 62 bp, synthetic construct
  source 1..62: mol_type = other DNA
gtctcgtgggctcggagatgtgtataagagacagcgaattacgattgcagcatcgggacgac

SEQ ID NO: 15 - DNA, 62 bp, synthetic construct
  source 1..62: mol_type = other DNA
gtctcgtgggctcggagatgtgtataagagacagattcttacgattgcagcatcgggacgac

SEQ ID NO: 16 - DNA, 62 bp, synthetic construct
  source 1..62: mol_type = other DNA
gtctcgtgggctcggagatgtgtataagagacagattgttacgattgcagcatcgggacgac

SEQ ID NO: 17 - DNA, 30 bp, synthetic construct
  source 1..30: mol_type = other DNA
tgagatttcttaaccaaaaaagttaagaca

SEQ ID NO: 18 - DNA, 30 bp, synthetic construct
  source 1..30: mol_type = other DNA
tgagattctaattaaataaataattagaca

SEQ ID NO: 19 - DNA, 30 bp, synthetic construct
  source 1..30: mol_type = other DNA
ttctgaattaaagagaataaatgaattgaa

SEQ ID NO: 20 - DNA, 30 bp, synthetic construct
  source 1..30: mol_type = other DNA
ttggttttataaaaacaaaagaaatgacaa

SEQ ID NO: 21 - DNA, 30 bp, synthetic construct
  source 1..30: mol_type = other DNA
gataactgagaataaatcgaattgtaaatc

SEQ ID NO: 22 - DNA, 30 bp, synthetic construct
  source 1..30: mol_type = other DNA
ttgaaaaaaacgaagtctttaaagattcaa

SEQ ID NO: 23 - DNA, 30 bp, synthetic construct
  source 1..30: mol_type = other DNA
ttctgaaagaatgaaaattctcgaattgaa

SEQ ID NO: 24 - DNA, 30 bp, synthetic construct
  source 1..30: mol_type = other DNA
ttaaaagctttaatcttaagattgaaataa

SEQ ID NO: 25 - DNA, 30 bp, synthetic construct
  source 1..30: mol_type = other DNA
ttacgaaaaaataacttaaatttttagtaa

SEQ ID NO: 26 - DNA, 30 bp, synthetic construct
  source 1..30: mol_type = other DNA
cagcattaattaagtaaataaaattaactg

SEQ ID NO: 27 - DNA, 30 bp, synthetic construct
  source 1..30: mol_type = other DNA
ttctgaattaaagggaataaatgaattgaa

SEQ ID NO: 28 - DNA, 30 bp, synthetic construct
  source 1..30: mol_type = other DNA
ttctgaattaaagaggataaatgaattgaa

SEQ ID NO: 29 - DNA, 30 bp, synthetic construct
  source 1..30: mol_type = other DNA
ttctgaattaaagagagtaaatgaattgaa

SEQ ID NO: 30 - DNA, 30 bp, synthetic construct
  source 1..30: mol_type = other DNA
ttctgaattaaagagaatgaatgaattgaa

SEQ ID NO: 31 - DNA, 30 bp, synthetic construct
  source 1..30: mol_type = other DNA
ttctgaattaaagagaatagatgaattgaa

SEQ ID NO: 32 - DNA, 30 bp, synthetic construct
  source 1..30: mol_type = other DNA
ttctgaattaaagagaataagtgaattgaa

SEQ ID NO: 33 - DNA, 30 bp, synthetic construct
  source 1..30: mol_type = other DNA
ttctgaattaaagagaacaaatgaattgaa

SEQ ID NO: 34 - DNA, 30 bp, synthetic construct; identical to SEQ ID NO: 19
  source 1..30: mol_type = other DNA

SEQ ID NO: 35 - DNA, 60 bp, synthetic construct
  source 1..60: mol_type = other DNA
cccatcagggggctaggctaacacggttcggctctctgagcccgggttatttcaggggga

SEQ ID NO: 36 - DNA, 12 bp, synthetic construct
  source 1..12: mol_type = other DNA
tgagattctaat

(19) United States
(12) Patent Application Publication
(10) Pub. No.: US 2026/0327629 A1
(43) Pub. Date: Oct. 22, 2026

(54) ENROFLOXACIN SPECIFIC APTAMERS AND USES THEREOF

(71) Applicant: City University of Hong Kong, Kowloon (HK)

(72) Inventors: Chun Kit KWOK, Kowloon (HK); Pik Kwan Peggy LO, Kowloon (HK); Sin Yu LAM, Kowloon (HK); Pan LI, Kowloon (HK)

(21) Appl. No.: 19/631,896

(22) Filed: Mar. 27, 2026

Related U.S. Application Data
(60) Provisional application No. 63/786,928, filed on Apr. 10, 2025.

(51) Int. Cl. C12N 15/115; G01N 21/64; G01N 33/18

(52) U.S. Cl. CPC C12N 15/115; G01N 21/643; G01N 33/1826; C12N 2310/16; G01N 2430/00

(57) ABSTRACT

An aptamer includes a polynucleotide sequence at least 90% identical to SEQ ID NO: 19, and a fluorescence molecule disposed at the 5′-end of the polynucleotide sequences. A method of detecting enrofloxacin in a sample. The method includes: (i) mixing the present aptamer with GO in a buffer solution to produce an aptasensor, in which the present aptamer is absorbed onto the GO; (ii) contacting the sample with the aptasensor; and (iii) measuring a fluorescent signal emitted from the fluorescent molecule disposed at the 5′-end of the present aptamer; wherein, an increase in the fluorescent signal in step (iii) indicates the presence of enrofloxacin in the sample.

### CROSS-REFERENCE TO RELATED APPLICATIONS

The present application claims priority to and the benefit of U.S. Provisional Patent Application No. 63/786,928 entitled “Enhanced Detection of Enrofloxacin in Seawater Using a Newly Selected Aptamer on a Graphite Oxide-Based Biosensor” and filed Apr. 10, 2025, which is hereby incorporated by reference in its entirety as if fully set forth below and for all applicable purposes.

### INCORPORATION BY REFERENCE OF MATERIAL IN XML FILE

This application incorporates by reference the Sequence Listing contained in the following XML file being submitted concurrently herewith: File name: 4702-15001_HP0379US_Sequence_List.xml, created on Mar. 17, 2026; and having a file size of 32.4 KB. The information in the Sequence Listing is incorporated herein in its entirety for all purposes.

### STATEMENT REGARDING PRIOR DISCLOSURES BY THE INVENTOR OR A JOINT INVENTOR UNDER 37 C.F.R. 1.77(B)(6)

Part of the subject matter of the invention described in the present application was published by the inventors, Chun Kit Kwok, Pik Kwan Lo, Sin Yu Lam and Pan Li in an article titled “Enhanced Detection of Enrofloxacin in Seawater Using a Newly Selected Aptamer on a Graphene Oxide-Based Biosensor”. The article was published in Anal. Chem. 2025, 97:6735-6744. The publication was made by and/or originated from 4 members of the inventive entity of the present invention, and the entirety of this article is incorporated herein by reference. A copy of the article is provided in a concurrently filed Information Disclosure Statement pursuant to the guidance of 78 Fed. Reg. 11076 (Feb. 14, 2013).”

### BACKGROUND OF THE INVENTION

### 1. Field of the Invention

The present disclosure in general relates to novel aptamers and uses thereof. Specifically, the present disclosure relates to aptamers that specifically recognize and bind to enrofloxacin, and methods for detecting residual environmental enrofloxacin by use of the present aptamers.

### 2. Description of Related Art

Enrofloxacin, a synthetic third-generation fluoroquinolone, is extensively used in veterinary medicine, particularly in aquaculture, to treat bacterial infections in fish and marine animals. However, its widespread use has led to the accumulation of enrofloxacin in the marine environment, raising significant ecological and public health concerns. Aquaculture discharge, containing residual antibiotics, enters rivers and eventually marine ecosystems, where enrofloxacin can persist and bioaccumulate in marine organisms like fish and shellfish, posing risks to food safety. Additionally, enrofloxacin disrupts natural microbial communities crucial for nutrient cycling and fosters the development of antibiotic-resistant bacteria, which can spread through the food chain and pose serious health risks to humans. Given these dangers, monitoring enrofloxacin levels in seawater is essential to protect marine ecosystems and public health. Currently, enrofloxacin residues in environmental samples have mainly been detected using traditional analytical techniques, including ultra-high performance liquid chromatography-tandem mass spectrometry (UPLC-MS/MS), high-performance liquid chromatograph-tandem mass spectrometry (HPLC-MS), gas chromatography-tandem mass spectrometry (GC-MS/MS), and enzyme-linked immunosorbent assay (ELISA).

While these methods provide high accuracy and sensitivity, they are not without significant limitations such as need for highly trained personnel, high-cost instrument, time-consuming, labor-intensive, and animal welfare concerns in ELISA. In light of these limitations, there is a pressing need to develop innovative, cost-effective, and user-friendly platforms for enrofloxacin analysis.

Aptamers are short (20-100 bases), single-stranded DNA or RNA oligonucleotides that exhibit high affinity and specificity for a wide range of targets, including small molecules, microorganisms, proteins, and cells. Due to their low cost, ease of chemical modification, high thermal stability, and straightforward generation, aptamers serve as biomolecular recognition elements in aptamer-based biosensors, known as aptasensors, for detecting small molecules. In 1990, an in vitro selection technique, Systematic Evolution of Ligands by Exponential Enrichment (SELEX) was first introduced to generate novel target binding aptamers. Multiple research groups have reported enrofloxacin-binding DNA aptamers with varying lengths and binding affinities for detecting in foods and lake water. To date, no studies have utilized Capture-SELEX for the selection of enrofloxacin-binding aptamers. Aptamers obtained from Capture-SELEX exhibit favorable structural switch properties, which have the potential to be applied in aptamer-based biosensor designs. However, aptamers alone generally face challenges of very poor limit of detection (LOD) when used as aptasensor, including issues with signal transduction, stability, shelf life, sensitivity, interference, and matrix effects.

To enhance detection performance, the use of aptamers in combination with various nanomaterials for signal transduction in biosensing is widely explored.

Nanomaterials such as magnetic nanoparticles, photon upconverting nanoparticles, gold nanoparticles (AuNPs), terbium (III) ions, and graphene/graphene oxide (GO), have been extensively investigated in conjugation with aptamers as recognition elements for the detection of enrofloxacin in real samples, including milk, fish, meat, lake water, river water, serum, urine, and honey. However, the fabrication of nanomaterial-based electrochemical aptasensors often requires multiple chemical conjugation steps, which can increase both the cost and time required for sensor development. These additional steps can complicate the overall process, making it less efficient and less feasible for large-scale or rapid testing applications. Furthermore, the analysis of complex real samples, such as seawater, poses additional challenges. Seawater contains a complex matrix of salts, organic matter, and various ions, which can interfere with the aggregation and disaggregation processes of gold nanoparticles commonly used in colorimetric detection. Such interference can lead to false positives or false negatives, reducing the reliability of the sensor. The presence of these interfering substances may compromise the sensitivity and accuracy of AuNP-based colorimetric assays, particularly in complex matrices where nonspecific interactions or aggregation can occur.

GO stands out among nanomaterials for environmental analysis due to its unique properties, including high electron transfer rates, large surface areas for recognition element immobilization, excellent electrical conductivity and fluorescence quenching capability, high aqueous solubility, stability in extreme pH, and inherent biocompatibility. GO can be synthesized in large quantities using simple and scalable methods, making it cost-effective for large-scale production. These attributes make GO more accessible and suitable than metallic nanoparticles for environmental monitoring applications. Despite its promising features, there are currently very limited studies that explore the use of GO-based detection platforms for the determination of enrofloxacin. A major obstacle is the nature of the reported enrofloxacin-binding aptamers, which consist of relatively long sequences (ranging from 60 to 80 nucleotides). These long sequences can result in strong, non-specific interaction to the GO surface, leading to challenges in sensor performance. Specifically, the tight binding of aptamers to the GO surface may prevent their complete detachment upon interaction with the target analyte, which hampers the desired fluorescence recovery signal in fluorescence-based detection assays. Additionally, the presence of redundant bases in these longer aptamer sequences may introduce steric hindrance, further reducing the aptamer's affinity and specificity for enrofloxacin. This issue is particularly problematic in complex matrices such as environmental samples, where nonspecific interactions with other substances can lead to false positive signals. As a result, these longer aptamers may exhibit moderate specificity and selectivity, limiting their effectiveness in real-world applications.

Accordingly, there exists in the related art a need for an improved aptamer that specifically binds to enrofloxacin and works with GO as an aptasensor to selectively detect residual enrofloxacin in seawater or wastewater.

### SUMMARY OF THE INVENTION

The present disclosure addresses the needs in the related art by providing novel aptamers specifically binds to enrofloxacin and methods for detecting environmental residual enrofloxacin by use of the present aptamers.

Accordingly, the first aspect of the present disclosure aims to provide a synthetic aptamer specifically binds to enrofloxacin. The synthetic aptamer comprises a polynucleotide sequence at least 90% identical to SEQ ID NO: 19, and a fluorescence molecule disposed at the 5′-end of the polynucleotide sequence.

According to embodiments of the present disclosure, the polynucleotide sequence is 100% identical to any one of SEQ ID NOs: 19, 27-31 or 33. Preferably, the polynucleotide sequence is 100% identical to SEQ ID NO: 19.

Examples of fluorescent molecule suitable for use in the present disclosure include, but are not limited to, carboxyfluorescein (FAM), 2′-chloro-7′-phenyl-1,4-dichloro-6-carboxy-fluorescein (VIC), 4,7,2′,4′,5′,7′-hexachloro-6-carboxy-fluorescein (HEX), 6-carboxy-4′-, 5′-dichloro-2′-, 7′-dimethoxy-fluorescein (JOE), 6-carboxytetramethyl-rhodamine (TMR), 2′-chloro-5′-fluoro-7′,8′-benzo-1,4-dichloro-6-carboxyfluorescein (NED), and 5- and 6-carboxy-X-rhodamine (ROX).

According to one preferred embodiment, the present synthetic aptamer comprises the polynucleotide sequence 100% identical to SEQ ID NO: 19 and the fluorescence molecule FAM disposed at the 5′-end of the polynucleotide sequence.

The second aspect of the present disclosure aims at providing an aptasensor for detecting enrofloxacin. The aptasensor comprises graphene oxide (GO), the present aptamer and a buffer solution, in which the present aptamer is absorbed onto the GO to form a GO-aptamer complex, which is suspended in the buffer solution.

According to preferred embodiments of the present disclosure, the present aptamer has a polynucleotide sequence 100% identical to any one of SEQ ID NOs: 19, 27-31 or 33. Preferably, the present aptamer has the polynucleotide sequence 100% identical to SEQ ID NO: 19. Examples of fluorescent molecule suitable for use in the present disclosure include, but are not limited to, FAM, VIC, HEX, JOE, TMR, NED, and

ROX.

According to one preferred embodiment, the present aptamer comprises the polynucleotide sequence 100% identical to SEQ ID NO: 19 and the fluorescence molecule FAM disposed at the 5′-end of the polynucleotide sequence.

According to preferred embodiments of the present disclosure, he buffer solution consists of 20 mM tris(hydroxymethyl)aminomethane hydrochloride (Tris-HCl), 20 mM KCl, 10 mM MgCl₂, 20 mM CaCl₂), 3% dimethyl sulfoxide (DMSO), and 0.05% polyoxyethylene (20) sorbitan monolaurate (Tween 20), and has a pH value of 7.5.

The third aspect of the present disclosure aims at providing a method of detecting enrofloxacin in a sample. The method comprises steps of:
(i) mixing the present aptamer with graphene oxide (GO) in a buffer solution to produce an aptasensor, in which the present aptamer is absorbed onto the GO;
(ii) contacting the sample with the aptasensor; and
(iii) measuring a fluorescent signal emitted from the fluorescent molecule disposed at the 5′-end of the aptamer of claim 1;
   wherein, an increase in the fluorescent signal in step (iii) indicates the presence of enrofloxacin in the sample.

According to embodiments of the present disclosure, the polynucleotide sequence is 100% identical to any one of SEQ ID NOs: 19, 27-31 or 33. Preferably, the polynucleotide sequence is 100% identical to SEQ ID NO: 19. Examples of the fluorescent molecule suitable for use in the present disclosure include, but are not limited to, FAM, VIC, HEX, JOE, TMR, NED, and ROX.

According to one preferred embodiment, the present aptamer comprises the polynucleotide sequence 100% identical to SEQ ID NO: 19 and the fluorescence molecule FAM disposed at the 5′-end of the polynucleotide sequence.

According to embodiments of the present disclosure, in step (i), the buffer solution consists of 20 mM tris(hydroxymethyl)aminomethane hydrochloride (Tris-HCl), 20 mM KCl, 10 mM MgCl₂, 20 mM CaCl₂), 3% dimethyl sulfoxide (DMSO), and 0.05% polyoxyethylene (20) sorbitan monolaurate (Tween 20), and has a pH value of 7.5.

According to embodiments of the present disclosure, the sample is seawater or wastewater.

The details of one or more embodiments of this disclosure are set forth in the accompanying description below. Other features and advantages of the invention will be apparent from the detailed descriptions, and from claims.

It is to be understood that both the foregoing general description and the following detailed description are by examples and are intended to provide further explanation of the invention as claimed.

### BRIEF DESCRIPTION OF THE DRAWINGS

The accompanying drawings, which are incorporated in and constitute a part of the specification, illustrate various example systems, methods and other exemplified embodiments of various aspects of the invention. The present description will be better understood from the following detailed description read in light of the accompanying drawings, where,

FIGS. 1A-1B Capture-SELEX sequence scheme for immobilization and positive selection steps. (FIG. 1A) The ssDNA N30 library was designed to be partially complementary to Biotin-capture oligo (Biotin-CO). The hybridized library pool was immobilized onto streptavidin coated magnetic beads (SA-MBs). (FIG. 1B) After removing the non-hybridized N30 library pool, enrofloxacin was introduced as the positive target to the beads. The binding between N30 library and the target induces structural switch, resulting in the dissociation of bound sequences from Biotin-TEG-CO.

FIGS. 2A-2B Characterization assays for the interaction between enrofloxacin and Enro_ap3 (aptamer). (FIG. 2A) Binding curve of Enro_ap3-FAM against enrofloxacin in binding buffer from the MST assay, and the K_{d} value of Enro_ap3 was found to be 1.64±0.181 μM. (FIG. 2B) The binding curve of Enro_ap3-FAM to ofloxacin showed no binding. The K_{d} values for the interactions of Enro_ap3-FAM-norfloxacin and Enro_ap3-FAM-tetracycline were determined to be 3.52±0.439 μM and 520÷297 μM, respectively.

FIG. 3 Specificity test of the fluorescent assay with enrofloxacin or other molecules. Enro_ap3 exhibited high selectivity towards other classes of antibiotics and moderate selectivity within the same class of antibiotics;

FIG. 4 is a schematic depiction illustrating the design and operation principles of the GO-aptasensor;

FIG. 5A is a line graph depicting the changes in fluorescence intensities of the GO-Enro_ap3-FAM platform upon introducing 10 μM enrofloxacin in Tris-HCl buffer over varying incubation periods;

FIG. 5B is a bar graph depicting the analysis of fluorescence spectra from the GO-Enro_ap3-FAM system following the addition of different enrofloxacin concentrations, ranging from 1 to 10 μM;

FIG. 5C depicts the assessment of fluorescence intensities for the GO-Enro_ap3-FAM detection platform subsequent to the introduction of several non-target antibiotics at a concentration of 10 μM. F₀ denotes the baseline fluorescence intensity of the GO-Aptasensor subsequent to the addition of sulfamethoxypyridine at a concentration of 0.2 μM. The GO-Enro_ap3-FAM platform concentration was maintained at 0.2 μM. Excitation occurred at a wavelength of 492 nm, with emission readings collected at 520 nm;

FIGS. 6A and 6B depict (FIG. 6A) the fluorescence intensities and (FIG. 6B) GO-Enro_ap3-FAM detection platform after spiking 10% seawater samples with varying concentrations of enrofloxacin (ranging from 0 to 100 μM), compared to the standard sample in Tris-HCl buffer system;

FIG. 6C depicts the fluorescence spectrum of Enro_ap3-FAM aptamers before (−GO) and after (+GO) the addition of GO substrates across different concentrations of seawater collected at Site 1; and

FIG. 6D depicts the fluorescence intensity of GO-Enro_ap3-FAM detection platform after the addition of 2 μL of enrofloxacin to solutions containing various ratios of seawater. The final concentration of Enro_ap3-FAM was 0.2 μM, with a total volume of 200 μL. Excitation wavelength: 492 nm, and the maximum emission collected was recorded at 520 nm.

### DETAILED DESCRIPTION OF THE PREFERRED EMBODIMENTS

The detailed description provided below in connection with the appended drawings is intended as a description of the present disclosure and is not intended to represent the only forms in which the present disclosure may be constructed or utilized.

### 1. Definitions

For convenience, certain terms employed in the specification, examples and appended claims are collected here. Unless otherwise defined herein, scientific and technical terminologies employed in the present disclosure shall have the meanings that are commonly understood and used by one of ordinary skill in the art.

Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “aptamer” as used herein refers to polynucleotide sequences that binds strongly to its target (e.g., an antibiotic or graphene oxide). According to embodiments of the present disclosure, the present aptamer may be constituted by DNAs alone, RNAs alone, or DNAs and RNAs in combination. Preferably, the aptamer of the present disclosure is a DNA aptamer about 30-mer in length. The present aptamer may intramolecularly comprise one double-stranded region and a region that is not base-paired between the strands. The “double-stranded region” refers to a region formed by consecutive base pairs between the nucleotide strands constituting the nucleic acid molecular. The length of the consecutive base pairs is 2 to 10 bp, such as 2 to 4 bp, 2 to 5 bp, 2 to 6 bp, 2 to 7 bp, 2 to 8 bp, 2 to 9 bp or 2 to 10 bp. The doubled stranded region is constituted by base pairs that are the same or different between the double-stranded regions, while the region that is not base-paired between the strands includes mismatch sites, gaps, or internal loop structures. According to embodiments of the present disclosure, the present aptamer exhibits affinity towards its target, which is a small molecule (i.e., an antibiotic or graphene oxide (GO)) that can serve as a target to which the aptamer may bind. According to embodiments of the present disclosure, in the present aptamer, the doubled-stranded region has an affinity toward GO, while the not base-paired region or the loop region has an affinity toward the antibiotic enrofloxacin. The length of each nucleotide strand of the present aptamer may range from 20 to 40 mer, such as 20, 21, 22, 23, 24, 25, 26, 27, 28, 29, 30, 31, 32, 33, 34, 35, 35, 37, 38, 39, or 40 mer; preferably 25 to 35 mer, such as 25, 26, 27, 28, 29, 30, 31, 32, 33, 34 or 35 mer; more preferably 30 mer. The base-paired strands do not have to have the same length, in other words, one strand may be longer or shorter than the other strand.

The term “sequence identity” as used herein, is generally expressed as a percentage and refers to the percent of nucleotides that are identical as between two sequences when optimally aligned. For the purposes of this invention, sequence identity means the sequence identity determined using the well-known Basic Local Alignment Search Tool (BLAST), which is publicly available through the National Cancer Institute/National Institutes of Health (Bethesda, Maryland). Percentage of sequence identity is calculated by comparing two optimally aligned sequences over a window of comparison, determining the number of positions at which the identical nucleic acid base occurs in both sequences to yield the number of matched positions, dividing the number of matched positions by the total number of positions in the window of comparison, and multiplying the result by 100 to yield the percentage of sequence identity. In the present disclosure, when a nucleic acid is described to be identical to another nucleic by certain percentage (e.g., 90% identical), it means the two nucleic acids share a sequence identity of that certain percentage (i.e., sequence identity of 90%). In some examples, the present aptamer has a polynucleotide sequence that is at least 90% identical to SEQ ID NO: 19, which means the present aptamer and the polynucleotide of SEQ ID NO: 19 share a sequence identity of at least 90%.

### 2. The Present Aptamer

The first objective of the present disclosure aims to provide an aptamer that binds strongly to its target, the antibiotic of enrofloxacin.

According to embodiments of the present disclosure, the aptamer comprises a polynucleotide sequence at least 90% identical to SEQ ID NO: 19, and a fluorescence molecule disposed at the 5′-end of the polynucleotide sequence.

According to embodiments of the present disclosure, the polynucleotide sequence is at least 90% identical to SEQ ID NO: 19, such as 90%, 91%, 92%, 93%, 94%, 95%, 96%, 97%, 98%, 99% or 100% identical to SEQ ID NO: 19. In some embodiments, the polynucleotide sequence is at least 96% identical to SEQ ID NO: 19, and is selected from the group consisting of SEQ ID NOs: 27, 28, 29, 30, 31 and 33. In one preferred embodiment, the polynucleotide sequence is 100% identical to SEQ ID NO: 19.

Examples of fluorescent molecule suitable for labeling the present aptamer include, but are not limited to, carboxyfluorescein (FAM), 2′-chloro-7′-phenyl-1,4-dichloro-6-carboxy-fluorescein (VIC), 4,7,2′,4′,5′,7′-hexachloro-6-carboxy-fluorescein (HEX), 6-carboxy-4′-, 5′-dichloro-2′-, 7′-dimethoxy-fluorescein (JOE), 6-carboxytetramethyl-rhodamine (TMR), 2′-chloro-5′-fluoro-7′,8′-benzo-1,4-dichloro-6-carboxyfluorescein (NED), and 5- and 6-carboxy-X-rhodamine (ROX). According to one preferred embodiment, the present aptamer is labeled with the fluorescence molecule FAM disposed at the 5′-end of the polynucleotide sequence of SEQ ID NO: 19.

According to embodiments of the present disclosure, the polynucleotide sequence of SEQ ID NO: 19 in the present aptamer is obtained through several runs of Capture-SELEX according to procedures described in the “Materials and methods” section, in which single-stranded DNA library is hybridized with a biotinylated capture oligonucleotide, the hybridized products are then immobilized onto streptavidin-coated magnetic beads, after positive and negative selections, the bound sequences on the magnet beads are eluted by placing them over a magnetic rack, the eluted products are subsequently amplified by PCR, and the PCR products are then regenerated and amplified to produce the aptamer of the present disclosure. According to embodiments of the present disclosure, the aptamer has the polynucleotide sequence 100% identical to SEQ ID NO: 19, which has a loop region that exhibits a specific binding affinity to enrofloxacin, and a double-stranded region that exhibits a non-specific binding affinity to graphene oxide (GO). Preferably, the specific binding affinity between the present aptamer of SEQ ID NO: 19 and enrofloxacin is about 1.64±0.181 μM.

According to optional embodiments of the present disclosure, the nucleotides in the loop region of the aptamer of SEQ ID NO: 19 may be mutated to produce variants. The thus produced variants independently have the polynucleotide sequences of SEQ ID NOs: 27, 28, 29, 30, 31 or 33, which exhibits a specific binding affinity to enrofloxacin of about 3.29±0.766 μM (SEQ ID NO: 27), 7.74±1.6 μM (SEQ ID NO: 28), 6.22±1.52 UM (SEQ ID NO: 29), 9.39±2.04 μM (SEQ ID NO: 30), 13.4±0.678 μM (SEQ ID NO: 31), or 5.20±1.31 μM (SEQ ID NO: 33).

According to embodiments of the present disclosure, each of the present aptamer described above exhibits a structural-switch property against enrofloxacin, in which the specific binding of the enrofloxacin to the loop region results in a structural change in the aptamer, thus the present aptamer could work with nanomaterial (e.g., GO), which exhibits non-specific binding to the present aptamer, for the detection of residual enrofloxacin present in an environmental sample (e.g., wastewater or sea water).

### 3. Use of the Present Aptamer

The second objective of the present disclosure aims at providing tools and methods for detecting residual environmental enrofloxacin by taking advantage of the binding affinities between the present aptamer and enrofloxacin or GO.

Accordingly, one aspect of the present disclosure aims to provide a sensor made from the present aptamer, i.e., aptasensor, which can detect environmental residual enrofloxacin. The aptasensor comprises GO, the present aptamer, and a buffer solution; in which the present aptamer is absorbed onto the GO to form a GO-aptamer complex, which is suspended in the buffer solution; and the buffer solution consists of 20 mM tris(hydroxymethyl)aminomethane hydrochloride (Tris-HCl), 20 mM KCl, 10 mM MgCl₂, 20 mM CaCl₂), 3% dimethyl sulfoxide (DMSO), and 0.05% polyoxyethylene (20) sorbitan monolaurate (Tween 20), and has a pH value of 7.5.

As the doubled-stranded region of the present aptamer exhibits a binding affinity to GO. Accordingly, upon mixing with GO, the double-stranded region of the present aptamer would be bond or physically absorbed onto the GO, thereby forming a GO-aptamer complex (i.e., the aptasensor). In such case, the selective TT-TT interactions and hydrogen bonding of the GO surface with the doubled-stranded region of the aptamer would result in quenching of the fluorescence emitted from the fluorophore of the present aptamer. However, the fluorescence quenching effect of GO is reversed when the present aptamer interacts with enrofloxacin, in such case, the binding of enrofloxacin to the loop region of the present aptamer causes a structural change, resulting in the previously absorbed aptamer to be released from the GO-aptamer complex. Once the aptamer is no longer bound with GO, the fluorescence quenching effect rendered by GO is relieved, allowing the aptamer to be detected again by the fluorescence emitted therefrom.

Accordingly, the present disclosure also encompasses a method of detecting residual enrofloxacin in the environment (e.g., seawater) by use of the aptasensor described above.

The method comprises steps of:
(i) mixing the present aptamer with GO in a buffer solution to produce an aptasensor, in which the present aptamer is absorbed onto the GO;
(ii) contacting the sample with the aptasensor of step (i); and
(iii) measuring a fluorescent signal emitted from the fluorescent molecule disposed at the 5′-end of the present aptamer;

wherein, an increase in the fluorescent signal in step (iii) indicates the presence of enrofloxacin in the sample.

According to embodiments of the present disclosure, the polynucleotide sequence is 100% identical to any one of SEQ ID NOs: 19, 27-31 or 33; and the fluorescent molecule is selected from the group consisting of FAM, VIC, HEX, JOE, TMR, NED, and ROX. Preferably, the present aptamer comprises the polynucleotide sequence 100% identical to SEQ ID NO: 19 and the fluorescence molecule FAM disposed at the 5′-end of the polynucleotide sequence.

According to embodiments of the present disclosure, the buffer solution consists of 20 mM tris(hydroxymethyl)aminomethane hydrochloride (Tris-HCl), 20 mM KCl, 10 mM MgCl₂, 20 mM CaCl₂), 3% dimethyl sulfoxide (DMSO), and 0.05% polyoxyethylene (20) sorbitan monolaurate (Tween 20), and has a pH value of 7.5.

According to embodiments of the present disclosure, in step (iii), the fluorescence emitted from the present aptamer increases linearly with an increase in the level of enrofloxacin in the sample, which is seawater. Furthermore, the detection is not affected by the salinity, acidity or total organic carbon (TOC) present in the seawater.

According to embodiments of the present disclosure, the level of detection (LOD) of the present aptamer on enrofloxacin is 32.15 μg/mL.

The present invention will now be described more specifically with reference to the following embodiments, which are provided for the purpose of demonstration rather than limitation. While they are typically of those that might be used, other procedures, methodologies, or techniques known to those skilled in the art may alternatively be used.

### EXAMPLES

### Materials and Methods

Capture-SELEX for DNA aptamers against enrofloxacin. The Capture-SELEX method was employed to select aptamers against enrofloxacin. The single-stranded DNA (ssDNA) initial library (for round 1) or ssDNA pool from previous rounds (for round 2 to 14) was designed to be partially complementary to the biotinylated capture strand (Biotin-TEG-CO). A volume of 250 μL of the mixture of libraries and the capture strand (1:2) was hybridized in SELEX buffer (200 mM NaCl, 10 mM MgCl₂, 6 mM KCl, 5 mM CaCl₂), 4% DMSO, and 40 mM Tris-HCl at pH 7.5) at 95° C. for 5 min, cooled to 25° C. for 10 minutes, and left at 4° C. overnight. Streptavidin-coated magnetic beads were vortexed and resuspended for 1 min, and the desired volume was transferred to a 1.5 ml tube. The ratio of magnetic beads (in μL) to ssDNA library pool (in pmol) was 1:5. The beads were washed three times with 500 μL of selection buffer. The hybridized DNA was immobilized onto the pre-washed magnetic beads and incubated for an hour at 25° C. The unbound library was discarded, and the beads were washed six times with 500 μL of selection buffer. Subsequently, the magnetic beads were incubated with a volume of 500 μL of enrofloxacin (ENRO) (positive target) in selection buffer at 25° C. for an hour for first round and 30 min for later rounds with shaking at 200 rpm. The eluted bound sequences from the magnetic beads were further concentrated and purified into the designed volume using RNA Clean & Concentrator™-5 (Zymo Research). The purified ssDNA was used as a template in the subsequent polymerase chain reaction (PCR) amplification. The PCR mix was composed of 1 μM of the forward primer, 1 μM of biotinylated reverse primer, 1× KAPA HiFi HotStart ReadyMix, and 1.5 μL of the template for a 20 μL reaction volume. The PCR amplification was carried out following the protocol of 95° C. for 3 min, (98° C. for 35 s, 55° C. for 25 s, 72° C. for 30 s) for n cycles, 72° C. for 45 s, and slow cooling to 4° C. (5° C./sec). The concentration of the PCR product was determined using 4200 TapeStation system (Agilent) using D1000 ScreenTape and buffer (Agilent). Subsequently, the desired volume of streptavidin-coated magnetic beads was pre-washed with selection buffer and then incubated with the dsDNA PCR product for an hour at 25° C. The unbound dsDNA was discarded, and the beads were washed on magnet five times with TEN250 (5M NaCl and 10× TE 7.4). To regenerate ssDNA from dsDNA, 250 μL of 0.2 μM sodium hydroxide was incubated at 25° C. for 25 min to break the hydrogen bonding. The incubated magnetic beads were placed on magnetic rack for 5 min. The eluted ssDNA was neutralized by adding 47 μL (tested with pH paper) of hydrochloric acid, followed by column purification using the RNA Clean & Concentrator™-5 (Zymo Research) kit according to the manufacturer's instruction. The collected ssDNA was used for next round of selection and for sample preparation for next-generation sequencing (NGS). The 5th, 7th, 9th, 11th, 13th, and 14th round of libraries underwent PCR using sequencing primers, which contained a unique 4-nt barcode (Table 1). The PCR product was concentrated and purified using Zymoclean Gel DNA Recovery Kits (Zymo Research). The eluted DNA was quantified using 4200 TapeStation system. A mix of 240 ng, containing 40 ng of the selected round of DNA, was dehydrated into a dry pellet using Concentrator plus (Eppendorf) and submitted for NGS sequencing. The sequencing results for the selected rounds were then analyzed.

TABLE 1
Sequences of oligonucleotides used for aptamer
selection and next-generation sequencing

| | | SEQ |
|---|---|---|
| | | ID |
| Name | Sequence (5′ - 3′) | NO |
| N30 | GGAGGCTCTCGGGACGAC(NNNNNNNNNN)₃GTCG | 1 |
| SSDNA | TCCCGATGCTGCAATCGTAA | |
| library | | |
| Forward | GGAGGCTCTCGGGACGAC | 2 |
| primer | | |
| (FP) | | |
| Biotin- | GTCGTCCCGAGAGCCATA-TEG-Biotin | 3 |
| capture | | |
| oligo | | |
| (Biotin- | | |
| CO) | | |
| Biotin- | Biotin-TTACGATTGCAGCATCGGGACG | 4 |
| reverse | | |
| primer | | |
| (Biotin- | | |
| RP) | | |
| NGS_FP1 | TCGTCGGCAGCGTCAGATGTGTATAAGAGACAGC | 5 |
| | CATGGAGGCTCTCGGGACGAC | |
| NGS_FP2 | TCGTCGGCAGCGTCAGATGTGTATAAGAGACAGC | 6 |
| | CAAGGAGGCTCTCGGGACGAC | |
| NGS_FP3 | TCGTCGGCAGCGTCAGATGTGTATAAGAGACAGC | 7 |
| | GATGGAGGCTCTCGGGACGAC | |
| NGS_FP4 | TCGTCGGCAGCGTCAGATGTGTATAAGAGACAGC | 8 |
| | GTTGGAGGCTCTCGGGACGAC | |
| NGS_FP5 | TCGTCGGCAGCGTCAGATGTGTATAAGAGACAGAT | 9 |
| | ACGGAGGCTCTCGGGACGAC | |
| NGS_FP6 | TCGTCGGCAGCGTCAGATGTGTATAAGAGACAGA | 10 |
| | TCCGGAGGCTCTCGGGACGAC | |
| NGS_RP1 | GTCTCGTGGGCTCGGAGATGTGTATAAGAGACAG | 11 |
| | CCTATTACGATTGCAGCATCGGGACGAC | |
| NGS_RP2 | GTCTCGTGGGCTCGGAGATGTGTATAAGAGACAG | 12 |
| | CCTTTTACGATTGCAGCATCGGGACGAC | |
| NGS_RP3 | GTCTCGTGGGCTCGGAGATGTGTATAAGAGACAG | 13 |
| | CGTATTACGATTGCAGCATCGGGACGAC | |
| NGS_RP4 | GTCTCGTGGGCTCGGAGATGTGTATAAGAGACAG | 14 |
| | CGAATTACGATTGCAGCATCGGGACGAC | |
| NGS_RP5 | GTCTCGTGGGCTCGGAGATGTGTATAAGAGACAG | 15 |
| | ATTCTTACGATTGCAGCATCGGGACGAC | |
| NGS_RP6 | GTCTCGTGGGCTCGGAGATGTGTATAAGAGACAG | 16 |
| | ATTGTTACGATTGCAGCATCGGGACGAC | |

^{a} A:T:G:C ratio in N30 region of ssDNA library is 25%:25%:25%:25%.
^{b} The NGS barcode is in bold font.

Microscale thermophoresis (MST) binding assay. The Monolith NT. 115 instrument (NanoTemper, Munich, Germany) was used for the the MST assay to assess the binding affinity and specificity between aptamer and its targets. The reaction mixture, totaling 10 μl, comprised 20 nM carboxyfluorescein (FAM)-labelled aptamer candidates and 1× SELEX buffer, which were denatured at 95° C. for 5 min and subsequently cooled to 4° C. Various concentrations of enrofloxacin ranging from 0.00763 μM to 250 μM were prepared through serial dilution. The mixture of FAM-labelled aptamer candidates (Enro_ap1-10) and antibiotics was then incubated at 25° C. for 30 min. FAM-labelled oligos were detected using the binding check mode with nano-blue excitation in Monolith Control Software at 25° C. The K_{d} values were determined using the MO. Affinity Analysis Software post-measurement.

For buffer salt dependence, concentrations of enrofloxacin ranging from 0.00381 to 125 μM was prepared in binding buffers bb01 to bb07 as listed in Table 2, and other procedures were the same. For binding test between Enro_ap3 and enrofloxacin, the same sample solution was prepared in bb07. Error bars represent the standard deviation from three independent replicates. GraphPad Prism 9 software was used to fit the curves with error bar and determine the K_{d} value.

TABLE 2
Composition of binding buffers

| | Tris- | | | | | | Tween | |
|---|---|---|---|---|---|---|---|---|
| | HCl | NaCl | KCl | MgCl₂ | CaCl₂ | DMSO | 20 | |
| | (mM) | (mM) | (mM) | (mM) | (mM) | (%) | (%) | pH |
| SELEX | 40 | 200 | 6 | 10 | 5 | 4 | — | 7.5 |
| buffer | | | | | | | | |
| bb01 | 20 | 200 | 20 | 10 | 20 | 3 | 0.05 | 7.5 |
| bb02 | 20 | 200 | 20 | 10 | — | 3 | 0.05 | 7.5 |
| bb03 | 20 | 200 | 20 | 2 | 20 | 3 | 0.05 | 7.5 |
| bb04 | 20 | 200 | 10 | 10 | 20 | 3 | 0.05 | 7.5 |
| bb05 | 20 | 100 | 20 | 10 | 20 | 3 | 0.05 | 7.5 |
| bb06 | 20 | 50 | 20 | 10 | 20 | 3 | 0.05 | 7.5 |
| bb07 | 20 | — | 20 | 10 | 20 | 3 | 0.05 | 7.5 |

^{a} Tris-HCl: Tris(hydroxymethyl)aminomethane hydrochloride.

For specificity test, all conditions remained the same except the targets, which changed from enrofloxacin to norfloxacin, ofloxacin, and tetracycline. For loop region of Enro_ap3 specificity test, FAM-labelled mutants (G14, G16, G17, C18, G19, G20, and G21)-enrofloxacin sample mixtures were prepared individually.

Strand displacement assay. FAM-labelled Enro_ap3 (100 nM) was hybridized and quenched with a 200 nM BHQ1-labelled strand in binding buffer at 95° C. for 5 min, 25° C. for 15 min, and 4° C. for at least 15 min. Subsequently, 10 μL of enrofloxacin at various concentrations was added to the resulting sensor complex in a total volume of 20 μL. The mixtures were then incubated at 25° C. for 30 min and cooled to 4° C. for 5 min. Following this, the mixtures were transferred to a 384-well black plate and monitored using a SpectraMax iD5 Standard Multi-Mode Microplate Reader (Ex: 485 nm; Em: 522 nm). Error bars represent the standard deviation from three independent replicates. GraphPad Prism 9 software was employed to fit the curves with error bar. In the specificity experiment in buffer, the final concentrations of enrofloxacin, norfloxacin, ofloxacin, sulfamethoxypyridine, tobramycin, oxytetracycline, and metronidazole were set at 100 μM.

Optimized the amount of GO in the quenching effect test. In this experiment, 2 μL of Enro_ap3-FAM capture probe with a concentration of 20 μM was added to various tubes containing different volumes of binding buffer (10 mM Tris-HCl, 1 mM KCl, 20 mM NaCl, 0.2 mM MgCl₂, pH 7.5): 188, 189, 190, 191, 192, 192.5, 193, 194, 195, 196, 197 and 198 μL. The fluorescence of each sample was then measured. Following this, varying amounts of GO were added to each corresponding tube in quantities of 10, 9, 8, 7, 6, 5.5, 5, 4, 3, 2, 1, and 0 μL. Subsequently, the fluorescence intensity of FAM was measured after 10 min to assess the quenching effect.

Fluorescence quenching effect over time. In the test that optimized the usage amount of GO to quench the Enro_ap3-FAM capture probe, 5.5 μL GO with a concentration of 0.1 mg/mL was determined as the appropriate volume. Subsequently, a fluorescence quenching efficiency study over time was conducted to investigate the quenching stability of GO with respect to FAM. 192.5 μL of binding buffer (10 mM Tris-HCl, 1 mM KCl, 20 mM NaCl, 0.2 mM MgCl₂, pH 7.5) was mixed with 2 μL of20 μM Enro_ap3-FAM capture probe for fluorescence measurement. Following this, 5.5 μL of a GO solution at a concentration of 0.1 g/mL was added, thoroughly mixed, and the fluorescence intensity of FAM was measured at various time points: 0, 2, 4, 6, 8, 10, 20, 30, 40, 50, 60 and 90 min.

Recovery effect test after addition of target into the GO-Enro_ap3-FAM nano platform. In a test tube, 2 μL of 20 μM Aptamer-FAM capture probe was added to 190.5 μL of a binding buffer (10 mM Tris-HCl, 1 mM KCl, 20 mM NaCl, 0.2 mM MgCl₂, pH 7.5) and mixed. Subsequently, 5.5 L of a GO solution with a concentration of 0.1 g/mL was added and thoroughly mixed to ensure complete quenching before fluorescence measurement. Following this, 2 μL enrofloxacin with a concentration of 1 mM was introduced, and the recovery fluorescence intensity of FAM was measured at various time points: 0, 1, 2, 3, 4, 5, 6, 7, 8, 9, 10, 12, 14, 16, 18, 20, 30, 40, 50, 60 and 90 min.

Specificity and selectivity tests. After the formation of the GO-Enro_ap3-FAM nanoplatform in the test tubes, 2 μL of 1 mM solutions of enrofloxacin, norfloxacin, ofloxacin, sulfamethoxypyridine, tobramycin, oxytetracycline, and metronidazole in DMSO were added to their respective tubes. Subsequently, the fluorescence intensity of FAM recovery was measured accordingly.

Detection in the spiked seawater samples. Four kinds of seawater from different locations were collected as real-time samples to assess the practical applications in seawater samples. The seawater samples were taken from local areas near Tai Ho Wan (seawater 1), Lung Kwu Sheung Tan (seawater 2), Wang Chau Kok (seawater 3), and Conic Island (seawater 4) and stored at 4° C. For the experimental procedure, a representative seawater sample (seawater 1) was selected. Enrofloxacin was diluted with ultrapure water to concentrations of 0, 0.2, 0.5, 1, 5, and 10 mM. Subsequently, 2 μL of each diluted enrofloxacin solution was added to 18 μL of the seawater, resulting in six spiked samples with final enrofloxacin concentrations of 0, 2, 5, 10, 50, 100 μM, each with a total volume of 20 μL. Each spiked sample was then mixed with 180 μL of binding buffer (10 mM Tris-HCl, 1 mM KCl, 20 mM NaCl, 0.2 mM MgCl₂, pH 7.5), which contained the GO quencher and the Aptamer-FAM capture probe. The fluorescence intensity of FAM was subsequently measured to assess the recovery efficiency. The same procedure was repeated for seawater samples 2, 3 and seawater 4.

To assess the influence of seawater on the GO-Enro_ap3-FAM nanoplatform, seawater 1 was selected as a representative sample. Initially, 2 μL of GO-Enro_ap3-FAM (20 μM) and 5.5 μL of GO (0.1 mg/mL) were mixed with varying volumes of seawater, specifically 0, 20, 40, 60, 80, 100, 120, 140, 160, 180, and 190.5 μL, and further combined with binding buffer to reach a total volume of 198 μL for each sample. Subsequently, 2 μL of enrofloxacin was added to each tested solution. Finally, fluorescence measurements were conducted.

Seawater Sample treatment for LC-MS/MS analysis. Briefly, 1 L of seawater sample was spiked with 50 μL of ofloxacin-d8 (1 μg/mL) as a surrogate. The pH of the filtered sample was adjusted to approximately 3 using formic acid, following which 0.2 g of Na2EDTA was added to chelate metals.

Solid phase extraction (SPE) was conducted for analyte extraction and cleanup. An OASIS HLB SPE cartridge (200 mg, 6 cc; Waters Corporation, MA, USA) was preconditioned with 5 mL of methanol, 5 mL of Milli-Q water, and 5 mL of 0.02% formic acid (FA) in Milli-Q water (v/v, pH≈3; referred to as acidified water hereafter). The water sample was then loaded onto the cartridge at a speed of 2 drops/s. After sample loading, the cartridge was washed with acidified water and then vacuum-dried at approximately 5 mmHg for 30 min. The analyte was eluted from the cartridge by adding 5 mL of methanol, with the eluate collected in a 15 mL polypropylene (PP) centrifuge tube. Subsequently, the eluate was evaporated under a gentle stream of high-purity nitrogen to dryness at 40° C. The final extract was reconstituted in 0.5 ml of 1:1 methanol/Milli-Q water (v/v) and transferred to an amber PP autosampler vial for instrumental analysis.

The determination of enrofloxacin was performed using an Agilent 1290 Infinity liquid chromatograph coupled with a SCIEX QTRAP 5500 tandem mass spectrometer (Woodlands, Singapore). The instrument operated with an electrospray ionization (ESI) interface in multiple-reaction monitoring (MRM) mode. Enrofloxacin was eluted from the liquid chromatograph (LC) through a 50 mm Agilent Zorbax Eclipse Plus C18 column with a 2.1 mm internal diameter and 1.8 μm particle diameter. The mobile phases used were Milli-Q water (0.02% FA) as mobile phase A and methanol (0.02% FA) as mobile phase B. Enrofloxacin was analyzed in the positive mode with the following electrospray ionization parameters: curtain gas, 35 psi; interface temperature, 300° C.; ion source gas 1, 50 psi; ion source gas 2, 50 psi; ionization voltage, 5000 V; and collision-associated dissociation (CAD) gas set to medium. Enrofloxacin detection was performed using multiple reaction monitoring mode.

In addition, seawater pH was measured using Eutech™ pH 700 Meter (Thermo Scientific, America). Seawater salinity was determined using the Salinity Meter AZ8373 (AZ Instrument Corp. China). Total organic carbon (TOC) in the seawater was analyzed using Elementar Vario Total Organic Carbon Analyzer fro, Elementar in Germany.

### Example 1

### Selection and Identification for Enrofloxacin Binding Aptamers

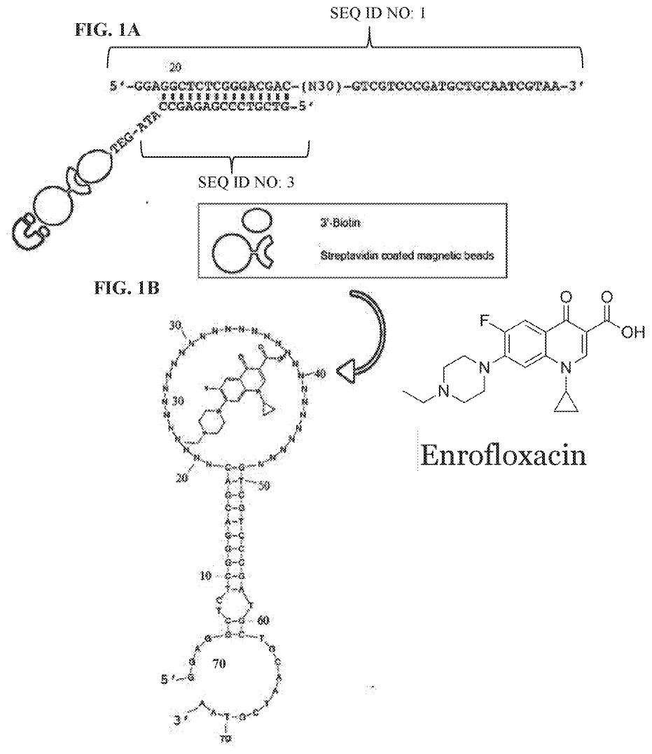

In this example, we refined the Capture-SELEX platform by employing streptavidin coated magnetic beads (SA-MB) as a solid matrix to immobilize the biotinylated hybridized library pool, which encoded an initial library with N30 random region and two primers that folded into a hairpin stem structure (FIG. 1). To ensure that the N30 ssDNA library was fully occupied, we used an excess of Biotin-TEG-CO (SEQ ID NO: 3) to hybridize with the un-labelled library. During positive selection, upon targeting binding, enrofloxacin-bound sequences were dissociated from the Biotin-TEG-CO, and the bound sequence elution was eluted by placing it in a magnetic rack.

To eliminate low affinity aptamer candidates from early rounds, we progressively increased the selection stringencies in each round. This included a decrease in the library pool from 1,000 μmol to 25 μmol and a reduction in the enrofloxacin concentration from 500 μM to 1 μM. To ensure sufficient enrichment of bound sequences, we conducted a total of 14 rounds of selection. Given that analogues within the same class of antibiotics share the same core structure but slightly different functional groups, we were concerned that the resulting aptamers might lack the target specificity, and thus we introduced ofloxacin as a counter target in the last 3 rounds before positive selection. Meanwhile, to prevent the loss of bound sequences during counter selection, we chose the ratio of positive target concentration to counter target concentration thoughtfully. From round 12 to 14, the ratio shifted from 10:1 to 1:1, and then to 1:10. After 14 rounds of selection, the ssDNA pool from rounds 5, 7, 9, 11, 13, and 14 were sequenced using next-generation sequencing (NGS).

From the NGS results, we assumed that the most enriched sequences from the final round would exhibit higher affinity and specificity due to the enhanced stringency of the reaction conditions during selection. Consequently, we selected the top 39 sequences for further analysis. During this analysis, we predicted their secondary structures using Mfold, a web server commonly used for the structural prediction of nucleic acids, identified structural motifs, and read percentage from NGS. Due to the target-dependent structural switching selection design, N30 sequences that exhibited the same secondary structure, both with and without fixed linker regions, were considered potential aptamer candidates. Next, from the entire sequencing results, we observed four main structural motifs that frequently appeared in less enriched sequences: “TTCTT”, “TGAGATTCTAAT (SEQ ID NO: 36)”, “GAATAAA”, and “AAGAAAAA”. Therefore, the sequences from top 39 sequences containing the same motifs were considered as potential aptamer candidates. Moreover, from earlier round to the last round, the sequences retained higher enrichment with increased reaction stringency. This trend suggested that the selection process effectively enriched for sequences that potentially bind to the target with high affinity, which was also one of the key analysis factors. Considering these factors, 10 aptamer candidates as listed in Table 3 were selected and named Enro_ap1 to Enro_ap10 for further validation.

TABLE 3
Sequences of aptamer candidates

| | | SEQ |
|---|---|---|
| | | ID |
| Name | Sequence (5′ - 3′) | NO |
| Enro_ap1- | FAM- | 17 |
| FAM | TGAGATTTCTTAACCAAAAAAGTTAAGACA | |
| Enro_ap2- | FAM- | 18 |
| FAM | TGAGATTCTAATTAAATAAATAATTAGACA | |
| Enro_ap3- | FAM- | 19 |
| FAM | TTCTGAATTAAAGAGAATAAATGAATTGAA | |
| Enro_ap4- | FAM- | 20 |
| FAM | TTGGTTTTATAAAAACAAAAGAAATGACAA | |
| Enro_ap5- | FAM- | 21 |
| FAM | GATAACTGAGAATAAATCGAATTGTAAATC | |
| Enro_ap6- | FAM- | 22 |
| FAM | TTGAAAAAAACGAAGTCTTTAAAGATTCAA | |
| Enro_ap7- | FAM- | 23 |
| FAM | TTCTGAAAGAATGAAAATTCTCGAATTGAA | |
| Enro_ap8- | FAM- | 24 |
| FAM | TTAAAAGCTTTAATCTTAAGATTGAAATAA | |
| Enro_ap9- | FAM-TTACGAAAAAATAACTTAAATTTTTA | 25 |
| FAM | GTAA | |
| Enro_ap10- | FAM- | 26 |
| FAM | CAGCATTAATTAAGTAAATAAAATTAACTG | |

### Example 2

### Aptamer Identification and Buffer Optimization for Enhancing Aptamer Binding

### 2.1 Aptamer Identification

To identify the binding affinity of the selected aptamer candidates of Example 1 (i.e., SEQ ID NOs: 17-26) for enrofloxacin, we employed microscale thermophoresis (MST) assay, measuring the changes in fluorescence as the fluorophore-labelled aptamer binds to enrofloxacin. A lower K_{d} value indicates a higher binding affinity between two molecules. Each aptamer candidates was tested individually in 1× SELEX buffer, yielding K_{d} values ranging from 10.1±2.76 μM to 232±171 μM. Specifically, the K_{d} values of Enro_ap3, Enro_ap4, Enro_ap6, Enro_ap7, Enro_ap8, and Enro_ap10 were determined to be 10.1±2.76 μM, 36.8±54.9 μM, 79.9±19.0 UM, 56.6±16.5 μM, 232±171 μM and 13.5±10.1 μM. Among the 10 aptamer candidates, Enro_ap3 (SEQ ID NO: 19) exhibited the highest binding affinity (K_{d}: 10.1±2.76 μM) and was therefore chosen for further optimization.

### 2.2 Buffer Optimization

Prior research indicated that salt concentration and buffer composition can significantly affect the conformational change of aptamers upon binding to the small molecules. To investigate the optimal binding conditions for the affinity between Enro_ap3 and enrofloxacin, we prepared and examined six different binding buffers (i.e., bb01 to bb06) respectively containing varying concentrations of potassium chloride (KCl), calcium chloride (CaCl₂)) and magnesium chloride (MgCl₂) using MST assay. The detailed compositions of each buffer were as listed in Table 2 in the “Material and methods” section.

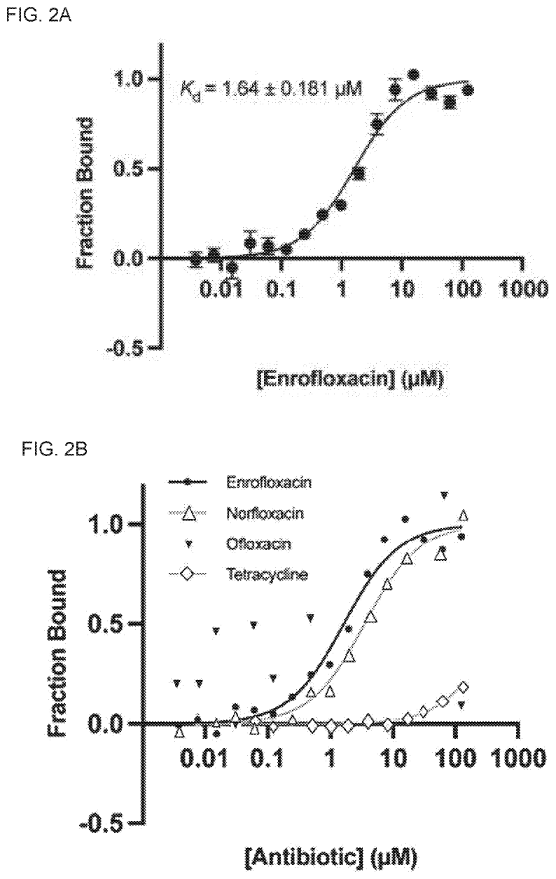

Initially, we incorporated Tween 20 into the buffers to reduce non-specific and enhance the solubility of enrofloxacin. The first modified buffer, designated bb01, exhibited a K_{d} value of 4.0±0.767 μM (FIG. 2A). This represented a significant improvement of approximately 2.5 times compared to the SELEX buffer by adjusting the concentration of enrofloxacin, as well as salt concentration and composition in the binding buffer (K_{d}: 10.1±2.76 μM). The

Following this, we optimized the concentrations of CaCl₂), MgCl₂, KCl, and NaCl based on the buffer composition of bb01. In the absence of CaCl₂), we observed no binding (i.e., bb01 vs bb02). Further, when the concentration of MgCl₂ was decreased to 2 mM, a slight increase in K_{d} value was observed (bb03: 6.22±0.955 μM). We concluded that higher concentrations of divalent salts might significantly improve the Enro_ap3-enrofloxacin interaction.

Furthermore, to investigate the effects of monovalent salts on binding affinity, we prepared buffers bb04, bb05, bb06 with lower KCl and lower NaCl, respectively (Table 2). Results indicated that higher concentrations of KCl and NaCl weakened the binding Enro_ap3 to the enrofloxacin (bb04: 3.90±0.610 μM, bb05: 6.22±0.955 μM, bb06: 1.8±0.287 μM). This underscores that the Enro_ap3-enrofloxacin is salt-dependent, providing valuable insights on the optimal buffer salt concentrations. Based on these observations we prepared a binding buffer bb07, which comprised 20 mM Tris-HCl, 20 mM KCl, 10 mM MgCl₂, 20 mM CaCl₂), 3% DMSO, 0.05% Tween 20, pH 7.5, for further analysis. It was found that the binding affinity improved 2.4-fold in this binding buffer bb07 compared to the bb01, achieving a K_{d} value of 1.64±0.181 μM. Thus, we employed this binding buffer bb07 for subsequent experiments.

### Example 3

### Evaluating the Specificity of the Identified Aptamer Enro_Ap3 of Example 2.1

To assess whether the identified aptamer Enro_ap3 of Example 2.1 did bind specifically to enrofloxacin, we evaluated its binding specificity by testing Enro_ap3 against non-targets using MST assay. Since ofloxacin served as counter target during the selection process, we observed there were arbitrary points shown in the MST curve, which indicated no binding. Additionally, we selected two other non-targets that were not involved in the selection process: norfloxacin as a representative from the same class of antibiotics and tetracycline as a representative from another class of antibiotics. Compared to Enro_ap3-enrofloxacin interaction (FIG. 2A), Enro_ap3 bound to norfloxacin with approximately 2-fold weaker binding affinity, yielding a K_{d} value of 3.52±0.439 μM (FIG. 2B). As different classes of antibiotics share different core structures, Enro_ap3 recognized and bound tetracycline with more than 300 times weaker affinity than enrofloxacin. This suggested that Enro_ap3 was highly specific to different classes of antibiotics while being moderately specific to the same class of antibiotics.

To delve into the loop region of aptamer specificity, we designed 7 aptamer constructs by single mutating the loop regions of Enro_ap3. Enro_ap3 is characterized as AT-rich, comprising of 80% both A and T bases. Therefore, we single-mutated the A bases in the loop region to G and the T bases to C. The resulting constructs as listed in Table 4 were designated as G14, G16, G17, C18, G19, G20, and G21, with K_{d} values ranging from 3.29±0.756 μM to 41.0±12.9 μM. These constructs exhibited weaker binding to enrofloxacin compared to Enro_ap3. From these experiments, we concluded that enrofloxacin specifically recognized the loop region of Enro_ap3, and thus we used Enro_ap3 for subsequent experiments

TABLE 4
Sequences of the 7 mutated aptamers.

| | | SEQ |
|---|---|---|
| | | ID |
| Name | Sequence (5′ - 3′) | NO |
| Enro_ap3_G14- | FAM- | 27 |
| FAM | TTCTGAATTAAAGGGAATAAATGAATTGAA | |
| Enro_ap3_G16- | FAM- | 28 |
| FAM | TTCTGAATTAAAGAGGATAAATGAATTGAA | |
| Enro_ap3_G17- | FAM- | 29 |
| FAM | TTCTGAATTAAAGAGAGTAAATGAATTGAA | |
| Enro_ap3_G19- | FAM- | 30 |
| FAM | TTCTGAATTAAAGAGAATGAATGAATTGAA | |
| Enro_ap3_G20- | FAM- | 31 |
| FAM | TTCTGAATTAAAGAGAATAGATGAATTGAA | |
| Enro_ap3_G21- | FAM- | 32 |
| FAM | TTCTGAATTAAAGAGAATAAGTGAATTGAA | |
| Enro_ap3_C18- | FAM- | 33 |
| FAM | TTCTGAATTAAAGAGAACAAATGAATTGAA | |

^{a} The mutated bases are in bold font.

### Example 4

### The Binding Property of the Aptamer Enro_ap3 in Strand Displacement Fluorescent Assay

In this example, a strand displacement fluorescent assay was used to investigate the binding property of the aptamer Enro_ap3 of Example 2.1, which was labelled with FAM fluorophore at the 5′-end. To quench the fluorescence of Enro_ap3-FAM, BHQ1 was labelled at the 3′-end of a 9-nt complementary strand designed to hybridize with Enro_ap3-FAM (SEQ ID NO: 19). In the presence of enrofloxacin, Enro_ap3 would bind to enrofloxacin and dissociate from the BHQ1-labelled strand, resulting in a recovery of fluorescence.

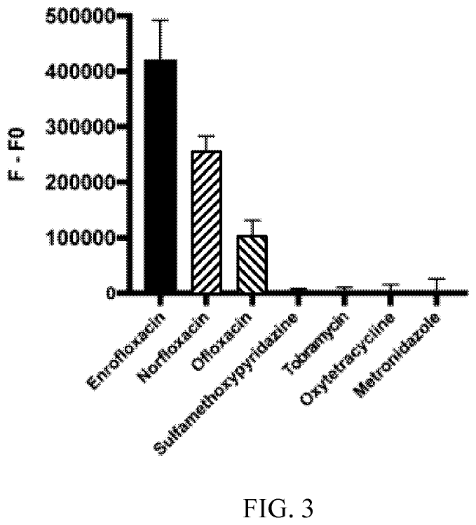

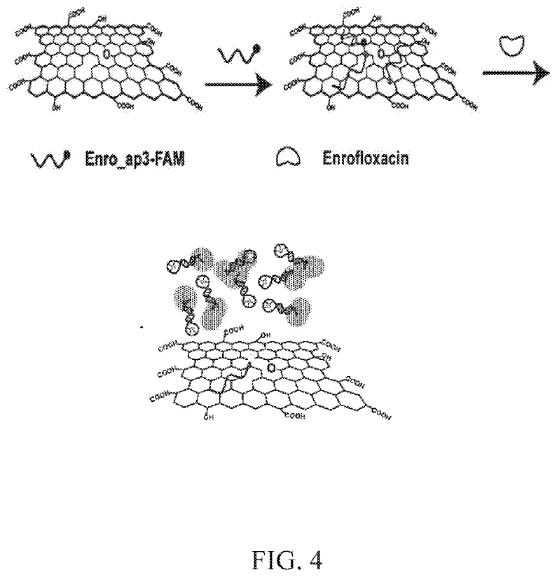

In this assay, we used an excess of the BHQ1-labelled strand (BHQ1-AATTCAGAA) (with FAM-Enro_ap3: BHQ1-labelled strand at a ratio of 1:2) to achieve a lower background and minimize false-positive results by ensuring that most of the FAM-Enro_ap3 was hybridized with the BHQ1. It was found the fluorescence intensity increased as varying concentrations of enrofloxacin were added, ranging from 500 nM to 2 mM. Before background subtraction, the fluorescence enhancement reached 8-fold after adding 2 mM of enrofloxacin. The response was linear from 1 to 100 μM enrofloxacin while the limit of detection (LOD) was found to be 86.1 μg/mL. To confirm the specificity of Enro_ap3 to enrofloxacin, we performed a similar set of experiment using 100 μM of 6 different antibiotics. This included antibiotics from the same class, such as norfloxacin and ofloxacin, as well as other classes, including sulfamethoxypyridine, tobramycin, oxytetracycline, and metronidazole. Due to the structural similarity, norfloxacin and ofloxacin were found to have the second and third highest fluorescence enhancement in this assay, while other classes of antibiotics remained at background level (FIG. 3)

### Example 5

### Construction of Graphene Oxide-based Aptasensor Using the Present Aptamer Enro_ap3

After confirming the binding ability of the present aptamer Enro_ap3 toward enrofloxacin, we then explored graphene oxide (GO) and aptamer-based platform as simple detection method and a strategy to minimize interference from seawater components while enhancing sensitivity and specificity in real-world applications. Accordingly, an aptasensor platform based on graphene oxide was created by combining FAM-labeled Enro_ap3 (Enro_ap3-FAM, SEQ ID NO: 19) as the recognition probe with GO acting as the fluorescence quencher in a Tris-HCl buffer. The functionality of GO-based biosensing platforms relies on the selective IT-IT interactions and hydrogen bonding of the GO surface with the aptamer, in preference to double-stranded nucleic acids or complex DNA structures. Upon interaction with the target antibiotic, the fluorescence quenching is reversed, serving as a detection signal, as the recognizing probes undergo conformational changes and detach from the surface of GO.

To optimize detection performance, fine-tuning of the amount of GO for effective fluorescence quenching was crucial in the development of aptasensor. A total of 2 μL of Enro_ap3-FAM capture probe, at a concentration of 20 μM, was mixed with varying concentration of GO solution. The results clearly demonstrated a gradual reduction in fluorescence intensity as the amount of GO increased. Even with just 1 μL of GO, the fluorescence intensity decreased by approximately 20% compared to the fluorescence intensity of the Enro_ap3-FAM capture probe alone. Remarkably, the quenching effect on FAM fluorescence reached nearly 95% when 5.5 μL of GO, at a concentration of 0.1 mg/mL, was added. Additionally, the quenching efficiency of GO showed a steady increase as more GO was introduced, ranging from 0 to 5.5 μL, revealing a nearly linear relationship between GO concentration and fluorescence quenching. The surface functional groups and size distribution of the GO substrate were characterized using Fourier Transform Infrared spectroscopy (FTIR) and transmission electron microscopy (TEM) analysis. Our findings confirmed that the binding between the aptamer and GO involved a multifaceted step, affected by the existence of both hydrophilic, highly oxidized regions, and carbon-rich, hydrophobic regions. These diverse interactions collectively contributed to the intricate adsorption behavior observed across various experimental settings. Based on these findings, 5.5 μL of GO at a 0.1 mg/mL concentration was selected as the optimized condition for quenching FAM fluorescence in subsequent experiments. The effect of fluorescence quenching as a function of incubation time was also investigated. It was found that rapid fluorescence quenching occurred within 1 min after adding of 5.5 μL GO to the Enro_ap3-FAM solution. Notably, this quenching effect remained stable over time without any increase in fluorescence intensity, indicating no fluctuations in the quenched state. This consistent behavior highlights the successful integration and stability of the GO-aptasensor detection system, establishing a solid foundation for its reliable performance as a biosensor.

### Example 6

### Detection of Enrofloxacin Using GO-aptasensor Nanoplatform

### 6.1 Detection by the Present GO-Aptasensor

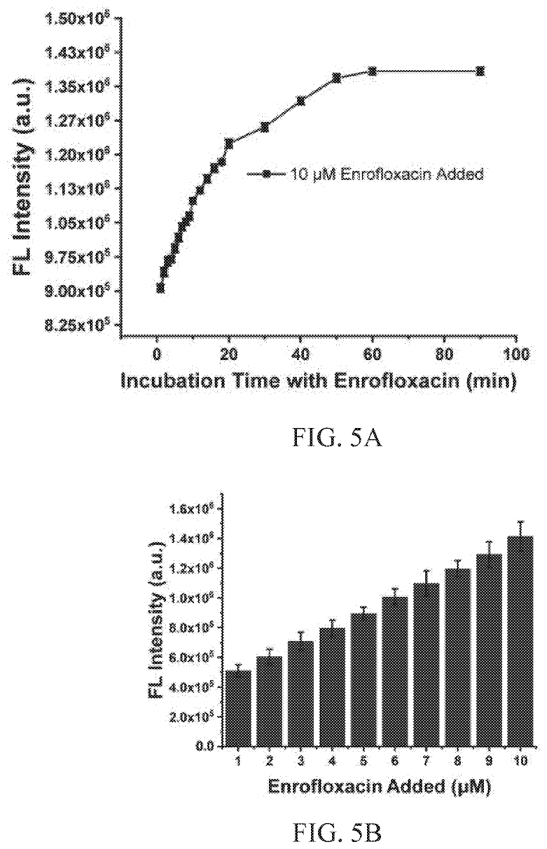

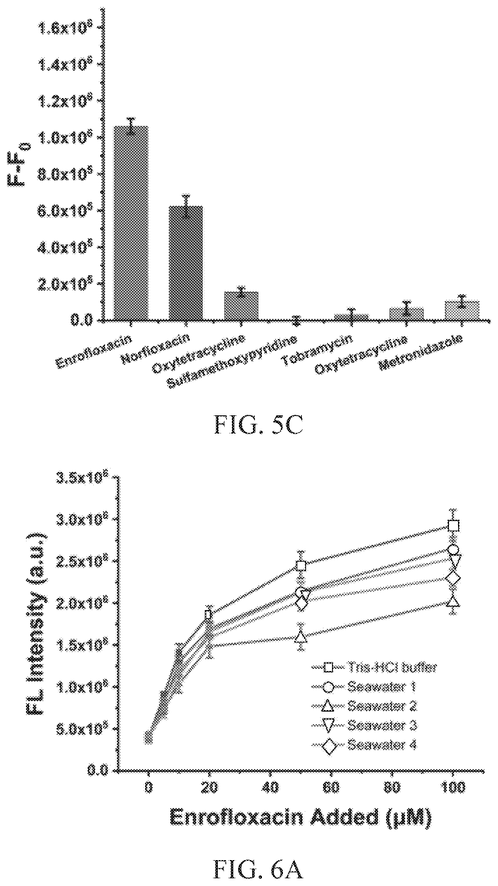

After optimizing the GO-aptasensor platform, its detection performance was investigated by measuring the fluorescence recovery upon the addition of the target antibiotic, enrofloxacin. It is found that the fluorescence intensity of the GO-Enro_ap3-FAM system progressively increased as a function of both enrofloxacin concentration and incubation time (FIG. 5A). During the initial 0-20 min of incubation, the fluorescence intensity displayed a nearly linear increase, indicating the rapid binding of enrofloxacin to the aptamer. Between 20 to 60 min, although the fluorescence intensity continued to rise, the rate of increase slowed down, eventually reaching a plateau around the 60-min. This suggested that the binding interaction between enrofloxacin and the aptamer approached saturation, and equilibrium was established within this time frame. The observed fluorescence recovery was primarily attributed to the specific binding interaction between enrofloxacin and the Enro_ap3-FAM. Upon binding to ENRO, the aptamer underwet a conformational change, folding into a specific secondary structure. This structural shift reduced its affinity for the GO surface, causing the aptamer to detach from the GO. As a result, the previously quenched fluorescence of the FAM dye (attached to the aptamer) was restored, leading to fluorescence recovery. Notably, the recovered fluorescence remained stable over time after reaching its maximum, demonstrating the robustness and reliability of the detection system. This robustness and stability were crucial for ensuring consistent and accurate detection in real-world applications. Moreover, the fluorescence intensity steadily increased with the addition of enrofloxacin across concentration ranges from 0 to 10 μM (FIG. 5B). A strong linear relationship was observed between enrofloxacin concentrations of 0 μM and 10 μM, indicating that the GO-aptasensor is highly responsive to change in enrofloxacin levels within this range. The limit of detection for enrofloxacin was calculated to be 32.15 μg/mL, based on the 3σ rule. This LOD value showed significant 2.5 times improvement compared to that of the original aptamer alone, highlighting the high sensitivity of the GO-aptasensor. This sensitivity makes it suitable for detecting trace amounts of enrofloxacin in complex samples such as environmental or biological matrices. Additionally, the excellent long-term stability of our newly developed GO-Enro_ap3-FAM confirmed its ability to maintain reliable detection performance even after prolong storage at room temperature.

### 6.2 Selectivity of the Present GO-Aptasensor

Selectivity is a critical factor for any target-specific aptasensor, ensuring that the sensor can distinguish the target molecules from other similar compounds. Although the selectivity of the Enro_ap3 aptamer has been previously validated, the selectivity of the entire GO-based aptasensor system requires further evaluation. To assess the specificity of the GO-aptasensor, we tested its response to a series of antibiotic from the same class or other classes. These antibiotics were chosen as negative controls to determine whether the system could distinguish enrofloxacin (ENRO) from other structurally or functionally similar compounds. As depicted in FIG. 5C, minimal fluorescence enhancement was observed for ofloxacin, sulfamethoxypyridine, tobramycin, oxytetracycline, and metronidazole, indicating low levels of non-specific binding or interference by these compounds. Although there was a noticeable signal obtained for NOR, its fluorescence intensity was still much lower than that of the target enrofloxacin. In contrast, the addition of enrofloxacin resulted in a pronounced fluorescence increase, approximately 3.5-fold higher than that observed with other tested antibiotics. This significant difference in fluorescence intensity confirmed the high specificity of the GO-based aptasensor for enrofloxacin. The system reliably recognized the target molecule, generating robust and distinct responses compared to non-target antibiotics. These results demonstrated that the GO-aptasensor was highly selective, capable of distinguishing enrofloxacin from a range of structurally diverse antibiotics. This high degree of selectivity was critical for real-world applications, where the presence of multiple contaminants may interfere with the detection of the target analyte. The strong signal differentiation observed here underscored the potential of this GO-aptasensor platform for specific and sensitive detection of enrofloxacin in complex samples.

### Example 7

### Detection of Enrofloxacin in Seawater Sample

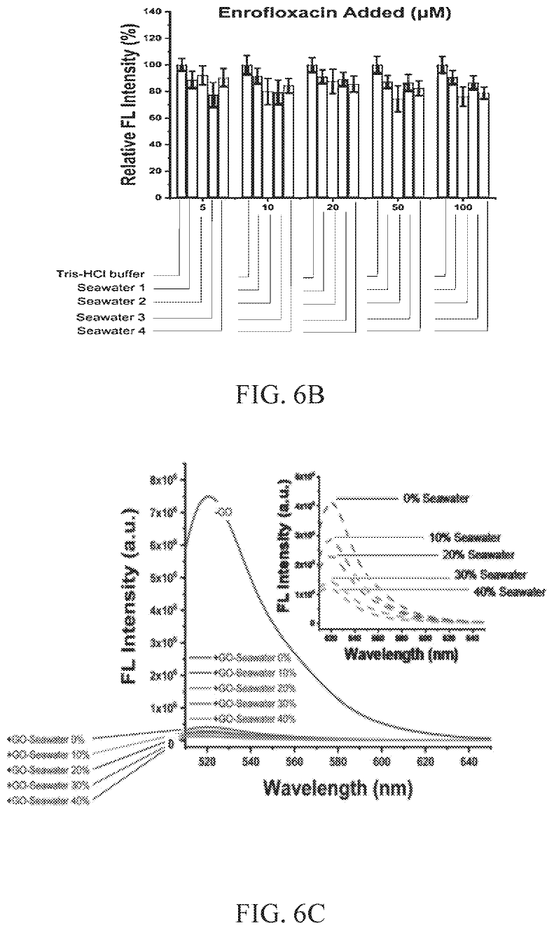

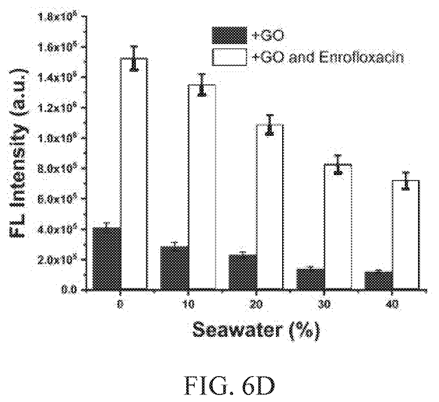

It is important to recognize that the flow of water and environmental factors can influence the salt concentrations and other components in seawater, this variability makes it crucial to investigate how different physiochemical parameters affect sensor's performance. In this study, we collected four types of seawater samples from distinct locations in Hong Kong to assess the performance of our GO-based aptasensor for detecting enrofloxacin. The sampling sites include areas near Tai Ho Wan (Site 1), Lung Kwu Sheung Tan (Site 2), Wang Chau Kok (Site 3), and Conic Island (Site 4). Seawater samples from Sites 1 and 2 exhibited relatively low salinity, with levels of 13 ppt and 10 ppt, respectively. The total organic carbon (TOC) concentrations in these samples were also low, with Site 1 showing 0.933±0.030 mg/mL and Site 2 showing 0.856±0.044 mg/L. In contrast, the seawater samples from site 3 and 4 demonstrated significantly higher salinity, at 28 and 30 ppt, respectively, and elevated TOC levels, with Site 3 containing 8.95±0.693 mg/L and Site 4 containing 11.8±0.432 mg/L. The pH values across all four sites were found to range from 7.49 to 7.91, which closely matched the pH value of the buffer used in the GO-aptasensor. These environmental parameters highlighted the variability in salinity and organic content across the different sites, which could potentially influence the sensor's performance. To evaluate the performance of the GO-based aptasensor in complex samples, we spiked 10% seawater samples with varying concentrations of enrofloxacin (ranging from 0 to 100 UM) and monitored the fluorescence intensity. As shown in FIG. 6A, the fluorescence intensity progressively increased with rising enrofloxacin concentration across all four seawater samples. This trend closely mirrored the control data obtained from the buffer system alone, suggesting that the aptasensor's performance was consistent, regardless of the seawater matrix. The recovery rates for enrofloxacin detection in seawater samples for sites 1, 2, 3 and 4 were also calculated, and ranged between 87%-92%, 74%-92%, 77%-90%, and 78%-90%, respectively (FIG. 6B). These results demonstrated that the GO-aptasensor was highly effective in detecting enrofloxacin in real seawater samples, despite variations in salinity and TOC. To further optimize the seawater content in the detection systems, we prepared solution with different seawater concentrations and tested them in the GO-aptasensor platform. FIG. 6C illustrates a noticeable fluorescence quenching effect when 5.5 μL of GO was added to the Enro_ap3-FAM solutions with varying seawater ratios. The fluorescence intensity consistently decreased as the seawater ratio increased, yet the quenching efficiency remained above 90% across all seawater concentrations. This consistent quenching efficiency highlighted GO's remarkable ability to quench Enro_ap3-FAM fluorescence, even in samples containing up to 40% seawater. A consistent trend of fluorescence recovery was achieved across all seawater concentrations, underscoring the sensor's strong detection capabilities and its potential for accurate enrofloxacin detection in complex seawater environments (FIG. 6D).

To compare the detection ability of the present GO-based aptasensor with other aptasensors in spiked seawater, two additional enrofloxacin binding aptamers respectively consisting of 37 and 60 bases as listed in Table 5 were created. These aptamers were independently modified with FMA fluorophores (i.e., “SEQ3-FAM” and “Ap17-FAM”) and were used as capture probes in combination with GO, forming “GO-SEQ3-FAM” and “GO-Ap17-FAM” aptasensors. These two aptamers could be effectively immobilized on the GO surface,

TABLE 5
Sequences of the enrofloxacin binding aptamers
with longer sequences.

| | | SEQ ID |
|---|---|---|
| Name | Sequence (5′ - 3′) | NO |
| SEQ3- | FAM-TTCTGAATTAAAGAGAATAAATGAATTGAA | 34 |
| FAM | | |
| Ap17- | FAM- | 35 |
| FAM | CCCATCAGGGGGCTAGGCTAACACGGTTCGGCTC | |
| | TCTGAGCCCGGGTTATTTCAGGGGGA | |

To further validate the accuracy of the developed sensors, we used the LC-MS/MS method to analyze the recoveries of enrofloxacin in spiked seawater collected at Site 1. The analytical characteristics of the enrofloxacin detection in seawater by the GO-aptasensor and LC-MS/MS (n=3) are listed in Table 6 and the results showed good agreement between the two methods. These findings demonstrated the analytical accuracy, reliability, and potential application of the present GO-aptasensor in real sample analysis.

TABLE 6
Detection of enrofloxacin in spiked seawater samples collected
at Site 1 by the developed GO-aptasensor and LC-MS/MS (n = 3)

| | GO-aptasensor | | | LC-MS/MS | | |
|---|---|---|---|---|---|---|
| | Measured | | | Measured | | |
| Spiked | Con. | Recovery | RSD | Con. | Recovery | RSD |
| Con. (μM) | (μM) | Rate (%) | (%, n = 3) | (μM) | Rate (%) | (%, n = 3) |
| 5 | 4.44 | 88.72 | 6.45 | 5.12 | 102.31 | 6.63 |
| 50 | 43.64 | 87.28 | 4.84 | 63.08 | 126.16 | 8.67 |
| 100 | 90.73 | 90.73 | 5.07 | 96.02 | 96.02 | 2.61 |

In sum, the present disclosure successfully selected a novel 30-mer enrofloxacin-binding aptamers via a refined Capture-SELEX platform, which led to a significant improvement in the construction of robust and reliable GO-aptasensor detection platform compared to the previously reported 60-mer enrofloxacin-binding aptamers. This shorter aptamer not only simplifies the design but also reduces strong non-specific binding to the GO surface, thereby enhancing its detection performance. The GO-aptasensor demonstrated high selectivity, effectively distinguishing enrofloxacin from a range of structurally diverse antibiotics. Its LOD for enrofloxacin was calculated to be 32.15 μg/mL, representing a significant 2.5-fold improvement compared to the original 30-mer aptamer alone, highlighting its high sensitivity. Compared to detection performance in binding buffer, the recovery rates in spiked seawater samples for enrofloxacin were within acceptable ranges. Importantly, the detection performance of GO-aptasensor was not influenced by variations in salinity, acidity, or total organic carbon concentrations in seawater samples collected. Comparable recovery rates were achieved between our GO-aptasensor platform and the traditional LC-MS/MS technique, indicating no significant effects on the sensor's performance in complex matrices. The results suggest that the GO-aptasensor is a promising tool for environmental monitoring and the detection of pharmaceutical contaminants in marine ecosystems.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of the present disclosure.

What is claimed is:

1. A synthetic aptamer specifically binds to enrofloxacin comprising a polynucleotide sequence at least 90% identical to SEQ ID NO: 19, and a fluorescence molecule disposed at the 5′-end of the polynucleotide sequence.

2. The synthetic aptamer of claim 1, wherein the polynucleotide sequence is 100% identical to any one of SEQ ID NOs: 19, 27-31 or 33.

3. The synthetic aptamer of claim 2, wherein the fluorescent molecule is selected from the group consisting of carboxyfluorescein (FAM), 2′-chloro-7′-phenyl-1,4-dichloro-6-carboxy-fluorescein (VIC), 4,7,2′,4′,5′,7′-hexachloro-6-carboxy-fluorescein (HEX), 6-carboxy-4′-, 5′-dichloro-2′-, 7′-dimethoxy-fluorescein (JOE), 6-carboxytetramethyl-rhodamine (TMR), 2′-chloro-5′-fluoro-7′,8′-benzo-1,4-dichloro-6-carboxyfluorescein (NED), and 5- and 6-carboxy-X-rhodamine (ROX).

4. The synthetic aptamer of claim 3, wherein the polynucleotide sequence is 100% identical to SEQ ID NO: 19 and the fluorescent molecule is the FAM.

5. An aptasensor for detecting enrofloxacin comprising graphene oxide (GO), the synthetic aptamer of claim 1, and a buffer solution,
wherein,
the synthetic aptamer is absorbed onto the GO to form a GO-aptamer complex, which is suspended in the buffer solution; and
the buffer solution consists of 20 mM tris(hydroxymethyl)aminomethane hydrochloride (Tris-HCl), 20 mM KCl, 10 mM MgCl₂, 20 mM CaCl₂), 3% dimethyl sulfoxide (DMSO), and 0.05% polyoxyethylene (20) sorbitan monolaurate (Tween 20), and has a pH value of 7.5.

6. The aptasensor of claim 5, wherein in the synthetic aptamer, the polynucleotide sequence is 100% identical to any one of SEQ ID NOs: 19, 27-31 or 33.

7. The aptasensor of claim 6, wherein in the synthetic aptamer, the fluorescent molecule is selected from the group consisting of FAM, VIC, HEX, JOE, TMR, NED and ROX.

8. The aptasensor of claim 7, wherein in the synthetic aptamer, the polynucleotide sequence is 100% identical to SEQ ID NO: 19 and the fluorescent molecule is the FAM.

9. A method of detecting enrofloxacin in a sample comprising:
(i) mixing the synthetic aptamer of claim 1 with GO in a buffer solution to produce an aptasensor, in which the synthetic aptamer is absorbed onto the GO;
(ii) contacting the sample with the aptasensor; and
(iii) measuring a fluorescent signal emitted from the fluorescent molecule disposed at the 5′-end of the aptamer;
wherein, an increase in the fluorescent signal in step (iii) indicates the presence of enrofloxacin in the sample.

10. The method of claim 9, wherein in the synthetic aptamer,
the polynucleotide sequence is 100% identical to any one of SEQ ID NOs: 19, 27-31 or 33; and
the fluorescent molecule is selected from the group consisting of FAM, VIC, HEX, JOE, TMR, NED and ROX.

11. The method of claim 10, wherein in the synthetic aptamer, the polynucleotide sequence is 100% identical to SEQ ID NO: 19 and the fluorescent molecule is the FAM.

12. The method of claim 9, wherein in step (i), the buffer solution consists of 20 mM tris(hydroxymethyl)aminomethane hydrochloride (Tris-HCl), 20 mM KCl, 10 mM MgCl₂, 20 mM CaCl₂), 3% dimethyl sulfoxide (DMSO), and 0.05% polyoxyethylene (20) sorbitan monolaurate (Tween 20), and has a pH value of 7.5.

13. The method of claim 9, wherein the sample is seawater or wastewater.

14. The method of claim 13, wherein the aptasensor can detect up to 32.15 μg enrofloxacin in 1 mL of the sample.
